(19) United States
(12) Patent Application Publication
(10) Pub. No.: US 2016/0347686 A1
(43) Pub. Date: Dec. 1, 2016

(54) METHOD OF STARTING UP A REACTOR FOR THE OXIDATIVE DEHYDROGENATION OF N-BUTENES

(71) Applicant: BASF SE, Ludwigshafen (DE)

(72) Inventors: Philipp GRÜNE, Mannheim (DE); Gauthier Luc Maurice AVERLANT, Darmstadt (DE); Ulrich HAMMON, Mannheim (DE); Ragavendra Prasad BALEGEDDE RAMACHANDRAN, Limburgerhof (DE); Jan Pablo JOSCH, Neustadt (DE); Christian WALSDORFF, Ludwigshafen (DE)

(73) Assignee: BASF SE, Ludwigshafen (DE)

(21) Appl. No.: 15/110,985

(22) PCT Filed: Jan. 9, 2015
(86) PCT No.: PCT/EP2015/050366

§ 371 Date: Jul. 12, 2016

(30) Foreign Application Priority Data
Jan. 13, 2014 (EP) 14150917.4

(51) Int. Cl. C07C 5/48

(52) U.S. Cl. CPC C07C 5/48

(57) ABSTRACT

The invention relates to a process for preparing butadiene from n-butenes having a start-up phase and an operating phase, wherein the process in the operating phase comprises the steps:

A) provision of a feed gas stream a1 comprising n-butenes;
B) introduction of the feed gas stream a1 comprising n-butenes, of an oxygen-comprising gas stream a2 and of an oxygen-comprising recycle gas stream d2 into at least one oxidative dehydrogenation zone and oxidative dehydrogenation of n-butenes to butadiene, giving a product gas stream b comprising butadiene, unreacted n-butenes, water vapor, oxygen, low-boiling hydrocarbons, high-boiling secondary components, possibly carbon oxides and possibly inert gases;
C) cooling and compression of the product gas stream b and condensation of at least part of the high-boiling secondary components, giving at least one aqueous condensate stream c1 and a gas stream c2 comprising butadiene, n-butenes, water vapor, oxygen, low-boiling hydrocarbons, possibly carbon oxides and possibly inert gases;
D) introduction of the gas stream c2 into an absorption zone and separation of incondensable and low-boiling gas constituents comprising oxygen, low-boiling hydrocarbons, possibly carbon oxides and possibly inert gases as gas stream d from the gas stream c2 by absorption of the C₄-hydrocarbons comprising butadiene and n-butenes in an absorption medium, giving an absorption medium stream loaded with C₄-hydrocarbons and the gas stream d, and recirculation, optionally after separating off a purge gas stream p, of the gas stream d as recycle gas stream d2 to the oxidative dehydrogenation zone;
and the start-up phase comprises the steps:
i) introduction of the oxygen-comprising gas stream and an inert gas stream into the dehydrogenation zone in such a ratio that the oxygen content of the recycle gas stream d2 corresponds to from 30 to 80% of the oxygen content of the recycle gas stream d2 in the operating phase;
ii) setting of the recycle gas stream d2 to at least 70% of the volume flow of the recycle gas in the operating phase;
iii) optional introduction, at an initial oxygen content of the recycle gas stream d2 of from 30 to 80% of the oxygen content of the recycle gas stream d2 in the operating phase, of a steam stream a3 into the dehydrogenation zone;
iv) introduction, at an initial oxygen content of the recycle gas stream d2 of from 30 to 80% of the oxygen content of the recycle gas stream d2 in the operating phase, of an oxygen-comprising gas stream a2′ and a butene-comprising feed gas stream a1′ having a smaller volume flow than in the operating phase in a ratio k=a2′/a1′ and raising of the volume flow of the gas streams a1′ and a2′ until the volume flows of the gas streams a1 and a2 in the operating phase are obtained, with the recycle gas stream d2 being at least 70% and not more than 120% of the volume flow in the operating phase.

The invention relates to a method of starting up a reactor for preparing 1,3-butadiene from n-butenes by oxidative dehydrogenation (ODH).

Butadiene is an important basic chemical and is used, for example, for producing synthetic rubbers (butadiene homopolymers, styrene-butadiene rubber or nitrile rubber) or for producing thermoplastic terpolymers (acrylonitrile-butadiene-styrene copolymers). Butadiene is also converted into sulfolane, chloroprene and 1,4-hexamethylenediamine (via 1,4-dichlorobutene and adiponitrile). Furthermore, it is possible to dimerize butadiene to produce vinylcyclohexene which can be dehydrogenated to styrene.

Butadiene can be prepared by thermal cracking (steam cracking) of saturated hydrocarbons, normally using naphtha as raw material. The steam cracking of naphtha gives a hydrocarbon mixture composed of methane, ethane, ethene, acetylene, propane, propene, propyne, allene, butanes, butenes, butadiene, butynes, methylallene, C₅— and higher hydrocarbons.

Butadiene can also be obtained by oxidative dehydrogenation of n-butenes (1-butene and/or 2-butene). Any mixture comprising n-butenes can be used as feed gas for the oxidative dehydrogenation (oxydehydrogenation, ODH) of n-butenes to butadiene. For example, it is possible to use a fraction which comprises n-butenes (1-butene and/or 2-butene) as main constituent and has been obtained from the C₄ fraction from a naphtha cracker by separating off butadiene and isobutene. Furthermore, gas mixtures which comprise 1-butene, cis-2-butene, trans-2-butene or mixtures thereof and have been obtained by dimerization of ethylene can also be used as feed gas. Gas mixtures which comprise n-butenes and have been obtained by fluid catalytic cracking (FCC) can also be used as feed gas.

The reaction of the gas streams comprising butenes is generally carried out industrially in shell-and-tube reactors which are operated in a salt bath as heat transfer medium. The product gas stream is cooled downstream of the reactor by direct contact with a coolant in a quenching stage and subsequently compressed. The C4 components are then absorbed in an organic solvent in an absorption column. Inert gases, low boilers, CO, CO₂ and others leave the column at the top. This overhead stream is partly fed as recycle gas to the ODH reactor. Hydrocarbons and oxygen can produce an explosive atmosphere. The concentration of combustible gas constituents (mainly hydrocarbons and CO) can be below the lower explosive limit (LEL) or above the upper explosive limit (UEL). Below the lower explosive limit, the oxygen concentration can be selected freely without an explosive gas mixture being able to form. However, the concentration of feed gas is then low, which is economically disadvantageous. A reaction with a reaction gas mixture above the upper explosive limit is therefore preferred. Whether an explosion can occur in this case depends on the oxygen concentration. Below a particular oxygen concentration, viz. the LOC (limiting oxygen concentration), the concentration of combustible gas constituents can be selected freely without an explosive gas mixture being able to form. LEL, UEL and LOC are temperature- and pressure-dependent.

On the other hand, precursors of carbonaceous material can be formed as a function of the oxygen concentration in the oxidative dehydrogenation of n-butenes to butadiene and these precursors of carbonaceous material can ultimately lead to carbonization, deactivation and irreversible destruction of the multimetal oxide catalyst. This is also possible when the oxygen concentration in the reaction gas mixture of the oxydehydrogenation at the inlet of the reactor is above the LOC.

The necessity of an excess of oxygen for such catalyst systems is generally known and is reflected in the process conditions when using such catalysts. As an example, reference may be made to the relatively recent studies of Jung et al. (Catal. Surv. Asia 2009, 13, 78-93; DOI 10.1007/s10563-009-9069-5 and Applied Catalysis A: General 2007, 317, 244-249; DOI 10.1016/j.apcata.2006.10.021).

However, the presence of high oxygen concentrations in addition to hydrocarbons such as butane, butene and butadiene or the organic absorbents used in the work-up section is associated with risks. Thus, explosive gas mixtures can form. If the reaction is carried out close to the explosive range, it is not always technically possible to prevent this range from being entered as a result of fluctuations in the process parameters. The time in which the reactor is started up and reaction gas mixture comes to flow through it is particularly critical in terms of the risk of explosion and carbonization of the catalyst.

Processes for the oxidative dehydrogenation of butenes to butadiene are known in principle.

US 2012/0130137A1, for example, describes a process of this type using catalysts which comprise oxides of molybdenum, bismuth and generally further metals. A critical minimum oxygen partial pressure in the gas atmosphere is necessary to avoid substantial reduction and thus a deterioration in performance of such catalysts and maintain long-term activity of the catalysts for the oxidative dehydrogenation. For this reason, it is generally also not possible to employ a stoichiometric input of oxygen or complete conversion of oxygen in the oxydehydrogenation reactor (ODH reactor). US 2012/0130137A1 describes, for example, an oxygen content of 2.5-8% by volume in the product gas.

In particular, the problems of formation of possible explosive mixtures after the reaction step are discussed in paragraph [0017]. In particular, it is indicated that in a “rich” mode of operation above the upper explosive limit in the reaction section there is the problem that after absorption of the major part of the organic constituents in the work-up, the gas composition crosses over into the explosive range with a change from a rich to a lean gas mixture. Thus, it is stated in paragraphs 0061-0062 that it is necessary according to the invention for the concentration of combustible gas constituents in the gas mixture fed to the oxidative dehydrogenation reactor to be above the upper explosive limit and on start-up of the oxidative dehydrogenation reaction for the oxygen concentration in the mixed gas at the reactor inlet to be initially set to a value below the limiting oxygen concentration (LOC) by firstly setting the amount of oxygen-comprising gas and steam introduced into the reactor and then starting the introduction of combustible gas (essentially feed gas). The amount of oxygen-comprising gas introduced, for example air, and combustible gas can subsequently be increased so that the concentration of combustible gas constituents in the mixed gas is above the upper explosive limit. As soon as the amount of combustible gas constituents and oxygen-comprising gas introduced increases, the amount of nitrogen and/or steam introduced is decreased in order to keep the amount of mixed gas introduced stable.

Furthermore, it is also pointed out that there is a risk of catalyst deactivation due to carbonization during an ongoing lean mode of operation in the reaction section. However, US 2012/0130137A1 does not give a solution to this problem.

In paragraph [0106] it is mentioned as an aside how the occurrence of explosive atmospheres in the absorption step can, for example, be avoided by dilution of the gas stream with nitrogen before the absorption step. In the more detailed description of the absorption step in paragraphs [132] ff, the problem of formation of explosive gas mixtures is not addressed further.

The document does not indicate the conditions which have to be adhered to in order to prevent carbonization of the catalyst. Furthermore, the document is not concerned with a process using the gas recycle mode of operation. Furthermore, the streams are set in succession, which means a high outlay for operation.

JP2010-280653 describes the starting of an ODH reactor. The reactor should be started without catalyst deactivation or an increase in the pressure drop occurring. This is said to be achieved by bringing the reactor to more than 80% of full load within 100 hours. In paragraph 0026, it is stated that, according to the invention, the amount of raw materials gas supplied to the reactor per unit time is set to more than 80% of the highest permissible amount to be supplied at the start of the reaction less than 100 hours after supply of the reactor with raw materials gas is commenced, and during this time the amount supplied of the nitrogen gas, of the gas comprising elemental oxygen and of steam introduced together with the raw materials gas into the reactor is regulated so that the composition of the mixed gas composed of raw materials gas, nitrogen gas, gas comprising elemental oxygen and steam does not get into the explosive range. The document does not describe the conditions which have to be adhered to in order to prevent carbonization of the catalyst. Furthermore the document does not relate to a process operated in the gas recycle mode. Furthermore, the document does not address the explosion problems in the work-up section of the process.

EP 1 180 508 describes the starting up of a reactor for catalytic gas-phase oxidation. Specifically, it describes the oxidation of propylene to acrolein. A process in which a range in which the oxygen content in the reaction gas mixture is greater than the LOC and the concentration of combustible gas constituents is below the LEL is gone through during start-up of the reactor is described. In steady-state operation, the O₂ concentration is then less than the LOC and the concentration of combustible gas constituents is greater than the UEL.

DE 1 0232 482 describes a process for the safe operation of an oxidation reactor for the gas-phase partial oxidation of propylene to acrolein and/or acrylic acid using a computer-aided switch-off mechanism. This is based on the recording of an explosion diagram in the computer memory and determination of the concentration of C₄ and O₂ by measurement of the O₂— and C₃-hydrdocarbon concentration in the recycle gas and the volume flow of recycle gas, O₃-hydrocarbon stream and oxygen-comprising gas. The starting up of the reactor is described in paragraphs 0076-0079. It is stated in paragraph 0079 that approval for opening the introduction of firstly air and then propene is only given when the inflow amount of the diluent gas (steam and/or recycle gas) has risen to a minimum value which is, for example, 70% of the maximum possible amount of air fed in. The concentration of O₂ in the recycle gas is identical to steady-state operation (3.3% by volume) even during the start-up process.

It is an object of the invention to provide a safe and economical method of starting up a reactor for the oxidative dehydrogenation of n-butenes to butadiene and also for starting up downstream units for the work-up of the product gas mixture.

The object is achieved by a process for preparing butadiene from n-butenes having a start-up phase and an operating phase, wherein the process in the operating phase comprises the steps:

A) provision of a feed gas stream a1 comprising n-butenes;

B) introduction of the feed gas stream a1 comprising n-butenes, of an oxygen-comprising gas stream a2 and of an oxygen-comprising recycle gas stream d2 into at least one oxidative dehydrogenation zone and oxidative dehydrogenation of n-butenes to butadiene, giving a product gas stream b comprising butadiene, unreacted n-butenes, water vapor, oxygen, low-boiling hydrocarbons, high-boiling secondary components, possibly carbon oxides and possibly inert gases;

C) cooling and compression of the product gas stream b and condensation of at least part of the high-boiling secondary components, giving at least one aqueous condensate stream c1 and a gas stream c2 comprising butadiene, n-butenes, water vapor, oxygen, low-boiling hydrocarbons, possibly carbon oxides and possibly inert gases;

D) introduction of the gas stream c2 into an absorption zone and separation of incondensable and low-boiling gas constituents comprising oxygen, low-boiling hydrocarbons, possibly carbon oxides and possibly inert gases as gas stream d from the gas stream c2 by substantial absorption of the C₄-hydrocarbons comprising butadiene and n-butenes in an absorption medium, giving an absorption medium stream loaded with C₄-hydrocarbons and the gas stream d, and recirculation, optionally after separating off a purge gas stream p, of the gas stream d as recycle gas stream d2 to the oxidative dehydrogenation zone;

and the start-up phase comprises the steps:

i) introduction of the oxygen-comprising gas stream and an inert gas stream into the dehydrogenation zone in such a ratio that the oxygen content of the recycle gas stream d2 corresponds to from 30 to 80% of the oxygen content of the recycle gas stream d2 in the operating phase;

ii) setting of the recycle gas stream d2 to at least 70% of the volume flow of the recycle gas d2 in the operating phase;

iii) optional introduction, at an initial oxygen content of the recycle gas stream d2 of from 30 to 80% of the oxygen content of the recycle gas stream d2 in the operating phase, of a steam stream a3 into the dehydrogenation zone;

iv) introduction, at an initial oxygen content of the recycle gas stream d2 of from 30 to 80% of the oxygen content of the recycle gas stream d2 in the operating phase, of an oxygen-comprising gas stream a2′ and a butene-comprising feed gas stream a1′ having a smaller volume flow than in the operating phase in a ratio k=a2′/a1′ and raising of the volume flow of the gas streams a1′ and a2′ until the volume flows of the gas streams a1 and a2 in the operating phase are obtained, with the recycle gas stream d2 being at least 70% and not more than 120% of the volume flow in the operating phase.

It has been found that the start-up method according to the invention enables a larger distance from the explosive limit to be maintained both in the oxydehydrogenation reactor (oxidative dehydrogenation zone, step B)) and in the quench (step C)) and in the C₄-hydrocarbon absorber (absorption zone, step D)). At the same time, carbonization of the catalyst during the start-up phase is avoided effectively.

In general, the ratio k is from 1 to 10, preferably from 1.5 to 6, in particular from 2 to 5. The ratio k is preferably essentially constant during the start-up phase, i.e. fluctuates by not more than ±50%, in particular by not more than ±20%.

In step ii), the recycle gas stream d2 is preferably set to from 80 to 120% of the volume flow in the operating phase. In a particularly preferred embodiment, the recycle gas stream d2 is set to 95-105% of the volume flow in the operating phase, with particular preference being given to the recycle gas stream d2 being set to 100% of the volume flow in the operating phase. The recycle gas stream d2 which has been set is kept essentially constant in the subsequent steps iii) and iv) and during the further start-up phase is at least 70% and not more than 120% of the volume flow of the recycle gas in the operating phase.

The oxygen content of the recycle gas stream d2 in step i) preferably corresponds to from 40 to 70%, in particular from 50 to 60%, of the oxygen content of the recycle gas stream d2 in the operating phase.

In a preferred embodiment of the invention, the introduction of the inert gas stream and of the oxygen-comprising gas is stopped between step i) and step ii).

In general, the amount of steam in the dehydrogenation zone during steps iii) and iv) is from 0 to 20% by volume, preferably from 1 to 10% by volume.

In general, the pressure in the dehydrogenation zone during the start-up phase is from 1 to 5 bar absolute, preferably from 1.05 to 2.5 bar absolute.

In general, the pressure in the absorption zone during the start-up phase is from 2 to 20 bar, preferably from 5 to 15 bar.

In general, the temperature of the heat transfer medium during the start-up phase is in the range from 220 to 490° C. and preferably from 300 to 450° C. and particularly preferably from 330 to 420° C.

In general, the duration of the start-up phase is in the range from 1 to 5000 minutes, preferably from 5 to 2000 minutes and particularly preferably from 10 to 500 minutes.

In general, step C) comprises the steps Ca) and Cb):

Ca) cooling of the product gas stream b in at least one cooling stage, where, in at least one cooling stage, cooling is effected by contacting with a coolant and condensation of at least part of the high-boiling secondary components;

Cb) compression of the remaining product gas stream b in at least one compression stage, giving at least one aqueous condensate stream c1 and a gas stream c2 comprising butadiene, n-butenes, water vapor, oxygen, low-boiling hydrocarbons, possibly carbon oxides and possibly inert gases.

In general, step D) comprises the steps Da) and Db):

Da) separation of incondensable and low-boiling gas constituents comprising oxygen, low-boiling hydrocarbons, possibly carbon oxides and possibly inert gases as gas stream d from the gas stream c2 by absorption of the C4-hydrocarbons comprising butadiene and n-butenes in an absorption medium, giving an absorption medium stream loaded with C4-hydrocarbons and the gas stream d, and

Db) subsequent desorption of the C4-hydrocarbons from the loaded absorption medium stream, giving a C4 product gas stream d1.

This is preferably followed by the additional steps E) and F):

E) separation of the C₄ product stream d1 by extractive distillation using a butadiene-selective solvent into a stream e1 comprising butadiene and the selective solvent and a stream e2 comprising n-butenes;

F) distillation of the stream f2 comprising butadiene and the selective solvent to give a stream g1 consisting essentially of the selective solvent and a butadiene-comprising stream g2.

In general, the gas stream d obtained in step Da) is recirculated to an extent of at least 10%, preferably at least 30%, as recycle gas stream d2 to step B).

In general, aqueous coolants or organic solvents or mixtures thereof are used in the cooling stage Ca).

An organic solvent is preferably used in the cooling stage Ca). This generally has a very much higher dissolution capability for the high-boiling secondary products, which in the parts of the plant downstream of the ODH reactor can lead to deposits and blockages, than water or alkaline aqueous solutions. Preferred organic solvents for use as coolant are aromatic hydrocarbons, for example toluene, o-xylene, m-xylene, p-xylene, diethylbenzenes, triethylbenzenes, diisopropylbenzenes, triisopropylbenzenes and mesitylene or mixtures thereof. Mesitylene is particularly preferred.

The embodiments below are preferred or particularly preferred variants of the process of the invention:

Stage Ca) is carried out in a plurality of stages in stages Ca1) to Can), preferably in two stages Ca1) and Ca2). Particular preference is given to at least part of the solvent which has gone through the second stage Ca2) being fed as coolant to the first stage Ca1).

Stage Cb) generally comprises at least one compression stage Cba) and at least one cooling stage Cbb). In the at least one cooling stage Cbb), the gas compressed in the compression stage Cba) is preferably brought into contact with a coolant. The coolant for the cooling stage Cbb) particularly preferably comprises the same organic solvent which is used as coolant in stage Ca). In a particularly preferred variant, at least part of this coolant which has gone through the at least one cooling stage Cbb) is fed as coolant to stage Ca).

Stage Cb) preferably comprises a plurality of compression stages Cba1) to Cban) and cooling stages Cbb1) to Cbbn), for example four compression stages Cba1) to Cba4) and four cooling stages Cbb1) to Cbb4).

Step D) preferably comprises the steps Da1), Da2) and Db):
Da1) absorption of the C₄-hydrocarbons comprising butadiene and n-butenes in a high-boiling absorption medium, given an absorption medium stream loaded with C₄-hydrocarbons and the gas stream d;
Da2) removal of oxygen from the absorption medium stream loaded with C₄-hydrocarbons from step Da) by stripping with an incondensable gas stream, and
Db) desorption of the C4-hydrocarbons from the loaded absorption medium stream, giving a C4 product gas stream d1 which consists essentially of C4-hydrocarbons and comprises less than 100 ppm of oxygen.

The high-boiling absorption medium used in step Da) is preferably an aromatic hydrocarbon solvent, particularly preferably the aromatic hydrocarbon solvent used in step Ca), in particular mesitylene. It is also possible to use, for example, diethylbenzenes, triethylbenzenes, diisopropylbenzenes and triisopropylbenzenes or mixtures comprising these substances.

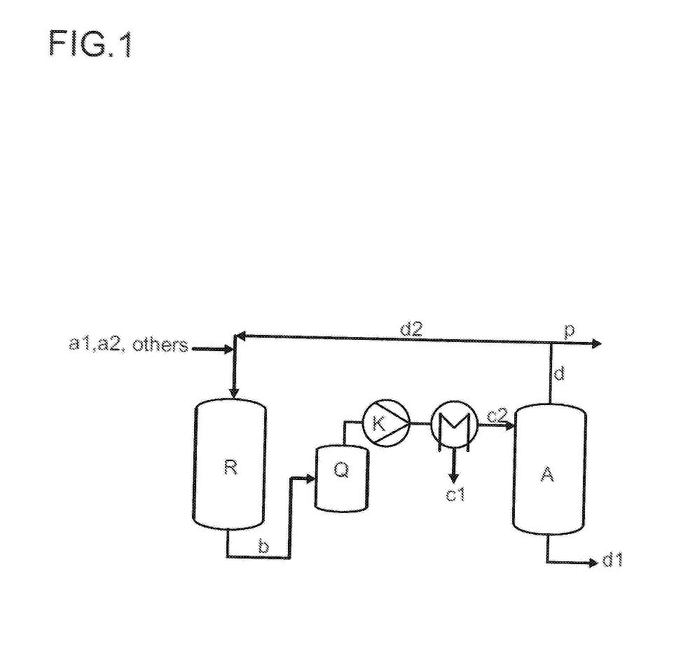

Embodiments of the process of the invention are shown in FIG. 1 and are described in detail below.

As feed gas stream, it is possible to use pure n-butenes (1-butene and/or cis-/trans-2-butene), or else gas mixtures comprising butenes. It is also possible to use a fraction which comprises n-butenes (1-butene and cis-/trans-2-butene) as main constituent and has been obtained from the C₄ fraction from the cracking of naphtha by separating off butadiene and isobutene. Furthermore, it is also possible to use gas mixtures which comprise pure 1-butene, cis-2-butene, trans-2-butene or mixtures thereof and have been obtained by dimerization of ethylene as feed gas. Gas mixtures which comprise n-butenes and have been obtained by fluid catalytic cracking (FCC) can also be used as feed gas.

In an embodiment of the process of the invention, the feed gas comprising n-butenes is obtained by nonoxidative dehydrogenation of n-butane. Coupling of a nonoxidative catalytic dehydrogenation with the oxidative dehydrogenation of the n-butenes formed makes it possible to obtain a high yield of butadiene, based on n-butane used. The nonoxidative catalytic dehydrogenation of n-butane gives a gas mixture which comprises butadiene, 1-butene, 2-butene and unreacted n-butane together with secondary constituents. Usual secondary constituents are hydrogen, water vapor, nitrogen, CO and CO₂, methane, ethane, ethene, propane and propene. The composition of the gas mixture leaving the first dehydrogenation zone can vary greatly as a function of the way in which the dehydrogenation is carried out. Thus, when the dehydrogenation is carried out with introduction of oxygen and additional hydrogen, the product gas mixture has a comparatively high content of water vapor and carbon oxides. In mode of operation without introduction of oxygen, the product gas mixture from the nonoxidative dehydrogenation has a comparatively high content of hydrogen.

In step B), the feed gas stream comprising n-butenes and an oxygen-comprising gas is fed into at least one dehydrogenation zone (the ODH reactor R) and the butenes comprised in the gas mixture are oxidatively dehydrogenated to butadiene in the presence of an oxydehydrogenation catalyst.

The gas comprising molecular oxygen generally comprises more than 10% by volume, preferably more than 15% by volume and even more preferably more than 20% by volume, of molecular oxygen. It is preferably air. The upper limit for the content of molecular oxygen is generally 50% by volume or less, preferably 30% by volume or less and even more preferably 25% by volume or less. In addition, any inert gases can be comprised in the gas comprising molecular oxygen. As possible inert gases, mention may be made of nitrogen, argon, neon, helium, CO, CO₂ and water. The amount of inert gases is generally 90% by volume or less, preferably 85% by volume or less and even more preferably 80% by volume or less, in the case of nitrogen. In the case of constituents other than nitrogen, it is generally 10% by volume or less, preferably 1% by volume or less.

To carry out the oxidative dehydrogenation at complete conversion of n-butenes, preference is given to a gas mixture which has a molar oxygen:n-butenes ratio of at least 0.5. Preference is given to working at an oxygen:n-butenes ratio of from 0.55 to 10. To set this value, the feed gas stream can be mixed with oxygen or at least one oxygen-comprising gas, for example air, and optionally additional inert gas or water vapor. The oxygen-comprising gas mixture obtained is then fed to the oxydehydrogenation.

Furthermore, inert gases such as nitrogen and also water (as water vapor) can be comprised in the reaction gas mixture. Nitrogen can serve for setting of the oxygen concentration and for preventing the formation of an explosive gas mixture, and the same applies to water vapor. Water vapor also serves for controlling carbonization of the catalyst and for removal of the heat of reaction.

Catalysts suitable for the oxydehydrogenation are generally based on an Mo—Bi—O-comprising multimetal oxide system, which generally additionally comprises iron. In general, the catalyst comprises further additional components such as potassium cesium, magnesium, zirconium, chromium, nickel, cobalt, cadmium, tin, lead, germanium, lanthanum, manganese, tungsten, phosphorus, cerium, aluminum or silicon. Iron-comprising ferrites have also been proposed as catalysts.

In a preferred embodiment, the multimetal oxide comprises cobalt and/or nickel. In a further preferred embodiment, the multimetal oxide comprises chromium. In a further preferred embodiment, the multimetal oxide comprises manganese.

Examples of Mo—Bi—Fe—O-comprising multimetal oxides are Mo—Bi—Fe—Cr—O— or Mo—Bi—Fe—Zr—O-comprising multimetal oxides. Preferred catalysts are, for example, described in U.S. Pat. No. 4,547,615 (Mo₁₂BiFe_{0.1}Ni₈ZrCr₃K_{0.2}Oₓ and Mo₁₂BiFe_{0.1}Ni₈AlCr₃K_{0.2}Oₓ), U.S. Pat. No. 4,424,141 (Mo₁₂BiFe₃Co_{4.5}Ni_{2.5}P_{0.5}K_{0.1}Oₓ+SiO₂), DE-A 25 30 959 (Mo₁₂BiFe₃Co_{40.5}Ni_{2.5}Cr_{0.5}K_{0.1}Oₓ, Mo_{13.75} BiFe₃Co_{4.5}Ni_{2.5}Ge_{0.5}K_{0.8}Oₓ, Mo₁₂BiFe₃Co_{4.5}Ni_{2.5}Mn_{0.5}K_{0.1}Oₓ and Mo₁₂BiFe₃Co_{4.5}Ni_{2.5}La_{0.5}K_{0.1}Oₓ), U.S. Pat. No. 3,911,039 (Mo₁₂BiFe₃Co_{4.5}Ni_{2.5}Sn_{0.5}K_{0.1}Oₓ), DE-A 25 30 959 and DE-A 24 47 825 (Mo₁₂BiFe₃Co_{4.5}Ni_{2.5}W_{0.5}K_{0.1}Oₓ).

Suitable multimetal oxides and their preparation are also described in U.S. Pat. No. 4,423,281 (Mo₁₂BiNi₈Pb_{0.5}Cr₃K_{0.2}Oₓ and Mo₁₂BibNi₇Al₃Cr_{0.5}K_{0.5}Oₓ), U.S. Pat. No. 4,336,409 (Mo₁₂BiNi₈Cd₂Cr₃P_{0.5}Oₓ), DE-A 26 00 128 (Mo₁₂BiNi_{0.5}Cr₃P_{0.5}Mg_{7.5}K_{0.1}Oₓ+SiO₂) and DE-A 24 40 329 (Mo₁₂BiCo_{4.5}Ni_{2.5}Cr₃P_{0.5}K_{0.1}Oₓ).

Particularly preferred catalytically active multimetal oxides comprising molybdenum and at least one further metal have the general formula (Ia):

Mo₁₂BiₐFe_{b}Co_{c}Ni_{d}CrₑX¹_{f}X²_{g}O_{y} (Ia),
where
X¹═Si, Mn and/or Al,
X²═Li, Na, K, Cs and/or Rb,
0.2≦a≦1,
0.5≦b≦10,
0≦c≦10,
0≦d≦10,
2≦c+d≦10
0≦e≦2,
0≦f≦10
0≦g≦0.5
y=a number determined by the valence and abundance of the elements other than oxygen in (Ia) so as to result in charge neutrality.

Preference is given to catalysts whose catalytically active oxide composition comprises only Co of the two metals Co and Ni (d=0). X¹ is preferably Si and/or Mn and X² is preferably K, Na and/or Cs, with particular preference being given to X²═K. A largely Cr(VI)-free catalyst is particularly preferred.

The catalytically active multimetal oxide composition can comprise chromium oxide. Possible starting materials are not only the oxides but also, especially, halides, nitrates, formates, oxalates, acetates, carbonates and/or hydroxides. The thermal decomposition of chromium(III) compounds into chromium(III) oxide occurs independently of the presence or absence of oxygen, mainly in the range 70-430° C., via a plurality of chromium(VI)-comprising intermediates (see, for example, J. Therm. Anal. Cal., 72, 2003, 135 and Env. Sci. Tech. 47, 2013, 5858). The presence of chromium(VI) oxide is not necessary for the catalytic oxydehydrogenation of alkanes to dienes, especially of butenes to butadiene. Owing to the toxicity and potential for damaging the environment of Cr(VI) oxide, the active composition should therefore be largely free of chromium(VI) oxide. The chromium(VI) oxide content depends largely on the calcination conditions, in particular the maximum temperature in the calcination step, and on the hold time in the calcination. The higher the temperature and the longer the hold time, the lower the content of chromium(VI) oxide.

The reaction temperature in the oxydehydrogenation is generally controlled by means of a heat transfer medium present around the reaction tubes. Possible liquid heat transfer media of this type are, for example, melts of salts or salt mixtures such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is in the range from 220 to 490° C. and preferably from 300 to 450° C. and particularly preferably from 330 to 420° C.

Owing to the exothermic nature of the reactions which proceed, the temperature in particular sections of the interior of the reactor during the reaction can be higher than that of the heat transfer medium and a hot spot is thus formed. The position and magnitude of the hot spot is determined by the reaction conditions, but can also be regulated by the dilution ratio of the catalyst bed or by passage of mixed gas. The difference between hot spot temperature and the temperature of the heat transfer medium is generally in the range 1-150° C., preferably 10-100° C. and particularly preferably 20-80° C. The temperature at the end of the catalyst bed is generally 0-100° C. above, preferably 0.1-50° C. above, particularly preferably 1-25° C. above, the temperature of the heat transfer medium.

The oxydehydrogenation can be carried out in all fixed-bed reactors known from the prior art, for example in a tray furnace, in a fixed-bed tube reactor or shell-and-tube reactor or in a plate heat exchanger reactor. A shell-and-tube reactor is preferred.

The oxidative dehydrogenation is preferably carried out in fixed-bed tube reactors or fixed-bed shell-and-tube reactors. The reaction tubes are (like the other elements of the shell-and-tube reactor) generally made of steel. The wall thickness of the reaction tubes is typically from 1 to 3 mm. Their internal diameter is generally (uniformly) from 10 to 50 mm or from 15 to 40 mm, frequently from 20 to 30 mm. The number of reaction tubes accommodated in the shell-and-tube reactor is generally at least 1000, or 3000, or 5000, preferably at least 10 000. The number of reaction tubes accommodated in the shell-and-tube reactor is frequently from 15 000 to 30 000 or up to 40 000 or up to 50 000. The length of the reaction tubes normally extends to a few meters; a typical reaction tube length is in the range from 1 to 8 m, frequently from 2 to 7 m, often from 2.5 to 6 m.

Furthermore, the catalyst bed installed in the ODH reactor R can consist of a single zone or of 2 or more zones. These zones can consist of pure catalyst or be diluted with a material which does not react with the feed gas or components of the product gas of the reaction. Furthermore, the catalyst zones can consist of all-active material and/or supported coated catalysts. The product gas stream leaving the oxidative dehydrogenation comprises not only butadiene but generally also unreacted 1-butene and 2-butene, oxygen and water vapor. As secondary components, it generally further comprises carbon monoxide, carbon dioxide, inert gases (mainly nitrogen), low-boiling hydrocarbons such as methane, ethane, ethene, propane and propene, butane and isobutane, possibly hydrogen and also possibly oxygen-comprising hydrocarbons, known as oxygenates. Oxygenates can be, for example, formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

The product gas stream at the reactor outlet has a temperature close to the temperature at the end of the catalyst bed. The product gas stream is then brought to a temperature of 150-400° C., preferably 160-300° C., particularly preferably 170-250° C. It is possible to insulate the line through which the product gas stream flows in order to keep the temperature in the desired range or to use a heat exchanger. This heat exchanger system can be of any type as long as this system enables the temperature of the product gas to be kept at the desired level. Examples of heat exchangers are spiral heat exchangers, plate heat exchangers, double-tube heat exchangers, multitube heat exchangers, vessel-spiral heat exchangers, vessel-wall heat exchangers, liquid-liquid contact heat exchangers, air heat exchangers, direct-contact heat exchangers and finned tube heat exchangers. Since part of the high-boiling by-products comprised in the product gas can precipitate while the temperature of the product gas is adjusted to the desired temperature, the heat exchanger system should preferably have two or more heat exchangers. If two or more heat exchangers provided are arranged in parallel and distributed cooling of the product gas in the heat exchangers is made possible, the amount of high-boiling by-products which deposit in the heat exchangers decreases and the period of operation of the heat exchangers can thus be increased. As an alternative to the above-described method, the two or more heat exchangers provided can be arranged in parallel. The product gas is fed to one or more, but not all, heat exchangers which after a particular period of operation are relieved by other heat exchangers. In this method, cooling can be continued, part of the heat of reaction can be recovered and, in parallel thereto, the high-boiling by-products deposited in one of the heat exchangers can be removed. As a coolant of the abovementioned type, it is possible to use a solvent as long as it is able to dissolve the high-boiling by-products. Examples are aromatic hydrocarbon solvents such as toluene and xylenes, diethylbenzenes, triethylbenzenes, diisopropylbenzenes, triisopropylbenzenes. Particular preference is given to mesitylene. It is also possible to use aqueous solvents. These can be made either acidic or alkaline, for example an aqueous solution of sodium hydroxide.

A major part of the high-boiling secondary components and of the water is subsequently separated off from the product gas stream by cooling and compression. Cooling is effected by contacting with a coolant. This stage will hereinafter also be referred to as quench Q. This quench can consist of only one stage or of a plurality of stages. The product gas stream is thus brought directly into contact with a preferably organic cooling medium and cooled thereby. Suitable cooling media are aqueous coolants or organic solvents, preferably aromatic hydrocarbons, particularly preferably toluene, o-xylene, m-xylene, p-xylene or mesitylene, or mixtures thereof. It is also possible to use all possible isomers of diethylbenzene, triethylbenzene, diisopropylbenzene and triisopropylbenzene and mixtures thereof.

Preference is given to a two-stage quench, i.e. the stage Ca) comprises two cooling stages Ca1) and Ca2) in which the product gas stream b is brought into contact with the organic solvent.

In a preferred embodiment of the invention, the cooling stage Ca) is thus carried out in two stages, with the solvent loaded with secondary components from the second stage Ca2) being fed into the first stage Ca1). The solvent taken off from the second stage Ca2) comprises a smaller amount of secondary components than the solvent taken off from the first stage Ca1).

A gas stream comprising n-butane, 1-butene, 2-butenes, butadiene, possibly oxygen, hydrogen, water vapor, small amounts of methane, ethane, ethene, propane and propene, isobutane, carbon oxides, inert gases and parts of the solvent used in the quench is obtained. Furthermore, traces of high-boiling components which have not been quantitatively separated off in the quench can remain in this gas stream.

The product gas stream from the solvent quench is compressed in at least one compression stage K and subsequently cooled further in the cooling apparatus, forming at least one condensate stream. A gas stream comprising butadiene, 1-butene, 2-butenes, oxygen, water vapor, possibly low-boiling hydrocarbons such as methane, ethane, ethene, propane and propene, butane and isobutane, possibly carbon oxides and possibly inert gases remains. Furthermore, this product gas stream can comprise traces of high-boiling components.

The compression and cooling of the gas stream can be carried out in one or more stages (in n stages). In general, the gas stream is compressed in total from a pressure in the range from 1.0 to 4.0 bar (absolute) to a pressure in the range from 3.5 to 20 bar (absolute). Each compression stage is followed by a cooling stage in which the gas stream is cooled to a temperature in the range from 15 to 60° C. The condensate stream can thus also comprise a plurality of streams in the case of multistage compression. The condensate stream comprises largely water and possibly the organic solvent used in the quench. Both streams (aqueous and organic phase) can also comprise small amounts of secondary components such as low boilers, C₄-hydrocarbons, oxygenates and carbon oxides.

The gas stream comprising butadiene, n-butenes, oxygen, low-boiling hydrocarbons (methane, ethane, ethene, propane, propene, n-butane, isobutane), possibly water vapor, possibly carbon oxides and possibly inert gases and possibly traces of secondary components is passed as output stream to further treatment.

In a step D), incondensable and low-boiling gas constituents comprising oxygen, low-boiling hydrocarbons (methane, ethane, ethene, propane, propene), carbon oxides and inert gases are separated off as gas stream from the process gas stream in an absorption column A by absorption of the C₄-hydrocarbons in a high-boiling absorption medium and subsequent desorption of the C₄-hydrocarbons. Step D preferably comprises the steps Da1), Da2) and Db):
Da1) absorption of the C₄-hydrocarbons comprising butadiene and n-butenes in a high-boiling absorption medium, giving an absorption medium stream loaded with C₄-hydrocarbons and the gas stream,
Da2) removal of oxygen from the absorption medium stream loaded with C₄-hydrocarbons from step Da) by stripping with an incondensable gas stream, giving an absorption medium stream loaded with C4-hydrocarbons and
Db) desorption of the C₄-hydrocarbons from the loaded absorption medium stream, giving a C₄ product gas stream consisting essentially of C₄-hydrocarbons.

For this purpose, the gas stream is brought into contact with an inert absorption medium in the absorption stage D) and the C₄-hydrocarbons are absorbed in the inert absorption medium, giving an absorption medium loaded with C₄-hydrocarbons and an offgas comprising the remaining gas constituents. In a desorption stage, the C₄-hydrocarbons are liberated again from the high-boiling absorption medium.

The absorption stage can be carried out in any suitable absorption column known to those skilled in the art. Absorption can be effected by simply passing the product gas stream through the absorption medium. However, it can also be carried out in columns or in rotary absorbers. Absorption can be carried out in cocurrent, countercurrent or cross-current. The absorption is preferably carried out in countercurrent. Suitable absorption columns are, for example, tray columns having bubble cap trays, centrifugal trays and/or sieve trays, columns having structured packing, e.g. sheet metal packing having a specific surface area of from 100 to 1000 m²/m³, e.g. Mellapak® 250 Y, and columns packed with random packing elements. However, trickle towers and spray towers, graphite block absorbers, surface absorbers such as thick film and thin film absorbers and also rotary absorbers, plate scrubbers, crossed spray scrubbers and rotary scrubbers are also possible.

In one embodiment, the gas stream comprising butadiene, n-butenes and the low-boiling and incondensable gas constituents is fed into the lower region of an absorption column. The high-boiling absorption medium is introduced in the upper region of the absorption column.

Inert absorption media used in the absorption stage are generally high-boiling nonpolar solvents in which the C₄-hydrocarbon mixture to be separated off has a significantly higher solubility than the remaining gas constituents to be separated off. Suitable absorption media are comparatively nonpolar organic solvents, for example aliphatic C₈-C₁₈-alkanes, or aromatic hydrocarbons such as the middle oil fractions from paraffin distillation, toluene or ethers having bulky groups, or mixtures of these solvents, with a polar solvent such as 1,2-dimethyl phthalate being able to be added to these. Further suitable absorption media are esters of benzoic acid and phthalic acid with straight-chain C₁-C₈-alkanols, and also heat transfer oils such as biphenyl and diphenyl ether, chloro derivatives thereof and triarylalkenes. One suitable absorption medium is a mixture of biphenyl and diphenyl ether, preferably in the azeotropic composition, for example the commercially available Diphyl®. This solvent mixture frequently comprises dimethyl phthalate in an amount of from 0.1 to 25% by weight.

In a preferred embodiment of the absorption stage Da1), the same solvent as in the cooling stage Ca) is used.

Preferred absorption media are solvents which have a dissolution capacity for organic peroxides of at least 1000 ppm (mg of active oxygen/kg of solvent). Preference is given to aromatic hydrocarbons, particularly preferably toluene, o-xylene, p-xylene and mesitylene and mixtures thereof. It is also possible to use all possible isomers of diethylbenzene, triethylbenzene, diisopropylbenzene and triisopropylbenzene and mixtures thereof.

At the top of the absorption column, a gas stream d which consists essentially of oxygen, low-boiling hydrocarbons (methane, ethane, ethene, propane, propene), the hydrocarbon solvent, possibly C₄-hydrocarbons (butane, butenes, butadiene), possibly inert gases, possibly carbon oxides and possibly also water vapor is taken off. This stream is at least partly recirculated as recycle gas stream d2 to the ODH reactor. This enables, for example, the feed stream to the ODH reactor to be set to the desired C₄-hydrocarbon content. In general, optionally after separating off a purge gas stream, at least 10% by volume, preferably at least 30% by volume, of the gas stream d is recirculated as recycle gas stream d2 to the oxidative dehydrogenation zone.

In general, the recycle stream amounts to from 10 to 70% by volume, preferably 30 to 60% by volume, based on the sum of all streams fed into the oxidative dehydrogenation B).

The purge gas stream can be subjected to a thermal or catalytic after-combustion. In particular, it can be thermally utilized in a power station.

At the bottom of the absorption column, residues of oxygen dissolved in the absorption medium are discharged by stripping with a gas in a further column. The proportion of oxygen which remains should be so small that the stream which leaves the desorption column and comprises butane, butene and butadiene comprises a maximum of 100 ppm of oxygen.

The stripping-out of the oxygen in step Db) can be carried out in any suitable column known to those skilled in the art. Stripping can be effected by simply passing incondensable gases, preferably gases such as methane which are not absorbable or only weakly absorbable in the absorption medium stream, through the loaded absorption solution. C4-hydrocarbons which are also stripped out are scrubbed back into the absorption solution in the upper part of the column by recirculating the gas stream into this absorption column. This can be achieved both by piping of the stripper column and also by direct mounting of the stripper column below the absorber column. Since the pressure in the stripping column part and absorption column part is identical, this direct coupling can be achieved. Suitable stripping columns are, for example, tray columns having bubble cap trays, centrifugal trays and/or sieve trays, columns having structured packing, e.g. sheet metal packing have a specific surface area of from 100 to 1000 m²/m³, e.g. Mellapak® 250 Y, and columns packed with random packing elements. However, trickle towers and spray towers and also rotary columns, plate scrubbers, cross-spray scrubbers and rotary scrubbers are also possible. Suitable gases are, for example, nitrogen or methane.

In an embodiment of the process, a methane-comprising gas stream is used for stripping in step Db). In particular, this gas stream (stripping gas) comprises >90% by volume of methane.

The absorption medium stream loaded with C₄-hydrocarbons can be heated in a heat exchanger and subsequently introduced into a desorption column. In one process variant, the desorption step Db) is carried out by depressurization and stripping of the loaded absorption medium by means of a steam stream.

The absorption medium which has been regenerated in the desorption stage can be cooled in a heat exchanger. The cooled stream comprises the absorption medium together with water which is separated off in a phase separator.

The C₄ product gas stream consisting essentially of n-butane, n-butenes and butadiene generally comprises from 20 to 80% by volume of butadiene, from 0 to 80% by volume of n-butane, from 0 to 10% by volume of 1-butene, from 0 to 50% by volume of 2-butenes and from 0 to 10% by volume of methane, where the total amount adds up to 100% by volume. Furthermore, small amounts of isobutane can be comprised.

Part of the condensed overhead output from the desorption column, which comprises mainly C4-hydrocarbons, can recirculated to the top of the column in order to increase the separation performance of the column.

The liquid or gaseous C4 product streams leaving the condenser can subsequently be separated by extractive distillation in step E) using a solvent which is selective for butadiene into a stream comprising butadiene and the selective solvent and a stream comprising butanes and n-butenes.

In a preferred embodiment of the process of the invention, a shutdown mechanism additionally prevents the oxidative dehydrogenation reactor from being supplied with a reaction gas mixture whose composition is explosive, where the shutdown mechanism is configured as follows:

a) an explosion diagram characteristic of the reaction gas mixture, in which explosive and nonexplosive compositions are delineated from one another as a function of the composition of the reaction gas mixture, is stored in a computer;

b) a data set is determined by measurements of the amount and optionally composition of the gas streams fed into the reactor for producing the reaction gas mixture and this data set is transmitted to the computer;

c) a current operating point of the reaction gas mixture in the explosion diagram is calculated by the computer from the data set obtained under b);

d) if the distance of the operating point from the closest explosion limit drops below a prescribed minimum value, the supply of gas streams to the reactor is automatically interrupted.

The minimum value is preferably calculated from a statistical error analysis of the measured parameters necessary for calculating the operating point.

The process allows heterogeneously catalyzed gas-phase partial oxidations and oxidative dehydrogenations of at least one organic compound to be carried out with increased safety at oxygen contents of the reaction mixture which are >=0.5, or >=0.75, or >=1, or >=2, or >=3, or >=5, or >=10, percentage points by volume above the limiting oxygen concentration. For the present purposes, the limiting oxygen concentration (LOC) is, as described above, the percentage by volume of molecular oxygen in the reaction gas mixture below which, independently of the quantity of the proportions by volume of the other constituents of the reaction gas mixture, namely, in particular, the organic compound to be oxidized and the inert diluent gas, a combustion (explosion) initiated by a local ignition source (e.g. local overheating or spark formation in the reactor) can no longer propagate spontaneously from the ignition source at the given pressure and temperature of the reaction gas mixture.

For safety reasons, it can be advantageous to record not the curve of the experimentally determined explosion limit as explosion diagram in a computer but instead to record a switching curve shifted downward therefrom by a safety margin. The safety margin is advantageously selected so that all error sources and measurement inaccuracies involved in the determination of the operating point of the reaction gas mixture are taken into account. The safety margin can be determined either by means of an absolute error analysis or by means of a statistical error analysis. In general, a safety margin of 0.1 to 0.4% by volume points of O₂ is sufficient.

Since the explosion behavior of butane and n-butenes is comparable and water vapor and nitrogen have a barely distinguishable effect on the explosion diagram of butane and/or butene, the following are, for example, possible as characteristic explosion diagram to be recorded in the computer according to the invention:

a) the butenes/O₂—N₂ diagram;

b) the butanes/O₂—N₂ diagram;

c) the butenes/O₂—H₂O diagram;

d) the butanes/O₂—H₂O diagram;

e) the butenes/O₂-(N₂/H₂O) diagram;

f) the butanes/O₂-(N₂/H₂O) diagram.

According to the invention, preference is given to recording the butenes/O₂—N₂ diagram in the computer.

In the experimental determination of the explosion diagram, a temperature which is not too far removed from the temperature range covered in the partial oxidation will be selected as temperature.

To calculate an informative current operating point of the reaction gas mixture in the explosion diagram, the experimental determination of, for example, the following parameters is sufficient:

a) the amount of air in standard m³ fed into the reaction per unit time;

b) the amount of butene-comprising feed gas in standard m³ fed into the reactor per unit time;

c) the amount of steam and/or recycle gas in standard m³ fed into the reactor per unit time;

d) the O₂ content of the recycle gas.

The oxygen and nitrogen contents of air are known, and the amount of feed gas comprising butenes and the amount of steam optionally also used are obtained as direct measurement results and the recycle gas is, apart from its oxygen content, assumed to consist exclusively of nitrogen. Should the recycle gas still comprise combustible constituents, this does not have a disadvantageous effect for the question of safety since their presence in the explosion diagram would merely shift the actual operating point to the right relative to the calculated operating point. Small amounts of water vapor or carbon oxides comprised in the recycle gas can be counted as nitrogen as far as safety relevance is concerned.

The measurement of the amounts of the gas streams fed into the reactor can be carried out using any measurement instrument suitable for this purpose. Possible measurement instruments of this type are, for example, all flow measurement instruments such as throttle instruments (e.g. orifice plates or Venturi tubes), displacement flow meters, float-type, turbine, ultrasonic, swirl and mass flow instruments. For reasons of the low pressure drop, Venturi tubes are preferred according to the invention. To take account of pressure and temperature, the measured volume flows can be converted into standard m³.

The determination of the oxygen content of the recycle gas can, for example, be carried out in-line as described in DE-A 10117678. It can, however, in principle also be carried out on-line by taking a sample from the product gas mixture coming from the oxidative dehydrogenation before it enters the target product isolation (work-up) and analyzed on-line in such a way that the analysis is effected in a period of time which is shorter than the residence time of the product gas mixture in the work-up. That is to say, the amount of gas supplied to the analytical instrument via an analysis gas bypass has to be appropriately large and the pipe system to the analytical instrument has to be appropriately small. It goes without saying that an O₂ determination could also be carried out on the reaction gas instead of the recycle gas analysis. Naturally, both can also be carried out. It is advantageous from a use point of view for the determination of the operating point for use of the safety-directed memory-programmed control (SMPC) to have an at least three-channel structure.

That is to say, each quantity measurement is carried out by means of three fluid flow indicators (FFI) connected in series or in parallel. The same applies to the O₂ analysis. If one of the three operating points calculated from the three data sets for the reaction gas mixture in the explosion diagram goes below the prescribed minimum safety margin, the gas inflow is automatically closed off in the order air, after a delay feed gas comprising butenes and finally, optionally steam and/or recycle gas.

From the point of view of a later restart, it can be advantageous to continue to circulate steam and/or recycle gas.

As an alternative, an average operating point in the explosion diagram can also be calculated from the three individual measurements. If the distance from this to the explosion limit goes below a minimum value, the above-described automatic shutdown takes place.

In principle, the method of the invention can be employed not only for steady-state operation but also for start-up and shutdown of the partial oxidation.

### EXAMPLES

The tube reactor (R) consists of stainless steel 1.4571, has an internal diameter of 29.7 mm and a length of 5 m and is filled with a mixed oxide catalyst (2500 ml). A thermocouple sheath (external diameter 6 mm) having a temperature sensor located within is installed in the center of the tube in order to measure the temperature profile in the bed. A salt melt flows around the tube in order to keep the exterior wall temperature constant. A stream composed of butenes and butanes (a1), steam, air and oxygen-comprising recycle gas is fed to the reactor. Furthermore, nitrogen can be fed to the reactor.

The offgas (b) is cooled in a quenching apparatus (Q) to 45° C., with the high-boiling by-products being separated off. The stream is compressed in a compressor stage (K) to 10 bar and cooled again to 45° C. A condensate stream c1 is discharged in the cooler. The gas stream c2 is fed to an absorption column (A). The absorption column is operated using mesitylene. A liquid stream rich in organic products and a gaseous stream d at the top of the absorption column are obtained from the absorption column. The overall work-up is designed so that water and the organic components are completely separated off. Part of the stream d is conveyed as recycle gas d2 back into the reactor.

### Example 1

The reactor and the work-up section are firstly flushed with a stream of 1000 standard l/h of nitrogen. After one hour, the measured oxygen content downstream of the reactor and in the recycle gas is less than 0.5% by volume. 240 standard l/h of air and 1000 standard l/h of nitrogen are then introduced into the reactor. The recycle gas stream is set to 2190 standard l/h. The recycle gas stream is kept constant by branching off an appropriately large purge gas stream downstream of the absorption column. After 20 minutes, the oxygen concentration in the recycle gas stream is 4.1% by volume. The supply of air and nitrogen to the reactor is stopped at the same time and 225 standard l/h of steam are fed into the reactor. Air and a stream consisting of 80% by volume of butenes and 20% by volume of butanes are then fed into the reactor, with the ratio of air flow to flow of butenes/butanes being regulated in such a way that this ratio is constant at about 3.75. Commencing with a flow of 44 standard l/h of butenes/butanes and 165 standard l/h of air, the flows are increased over a period of one hour at a constant ramp and after on hour are 440 standard l/h of butenes/butanes and 1650 standard l/h of air. The recycle gas stream is kept constant during the entire start-up operation by separating off an appropriate purge gas stream and is 2190 standard l/h.

The butenes are reacted to an extent of 83% at a salt bath temperature of 380° C. The selectivity of the conversion of butenes into butadiene is 92%, that into CO and CO₂ together is 5% and that into other secondary components is 3%.

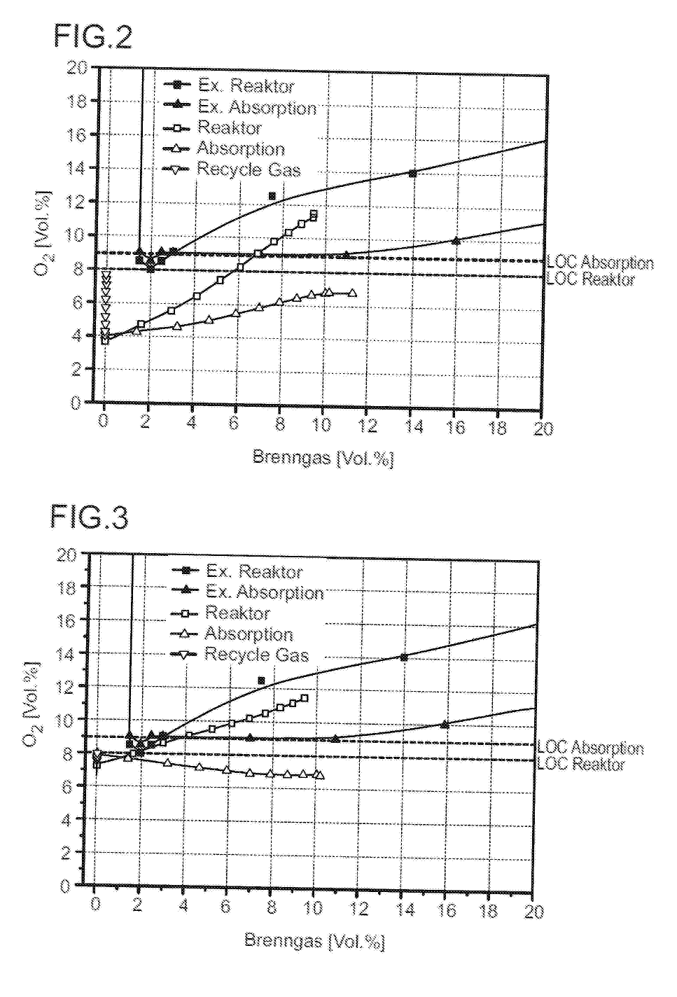

The plant is operated for 4 days and a steady state in which the concentrations of the gas components change by not more than 5%/h is established. The concentrations in the steady state upstream and downstream of the reactor and also in the recycle gas are shown in table 1. The concentration curves for butanes/butenes (fuel gas), oxygen and the remaining gas components (100%−c_{fuel gas}−c_{O2}) upstream of the reactor (“reactor”), and between the quench and the compression stage (“absorption”) and in the recycle gas (“recycle gas”) is shown together with the explosion diagrams for the reactor (“ex. reactor”) and the absorption column (“ex. absorption”) in FIG. 2. All concentrations are reported in percent by volume. The concentration of the fuel gas is plotted on the ordinate, and the concentration of oxygen is plotted on the abscissa. Immediately before the introduction of fuel gas (butenes and butanes) is commenced, the oxygen concentration upstream and downstream of the reactor, between the quench and absorption column and in that recycle gas is 4.1% by volume. During the time in which the fuel gas flow is increased to the final value, the oxygen concentration in the recycle gas increases to a final value of about 7.6% by volume. The oxygen concentration also increases upstream of the reactor and between the quench and the absorption column, but without crossing into the explosive region. Safe start-up can thus be ensured.

TABLE 1

| | Reactor inlet | Reactor outlet | Recycle gas |
|---|---|---|---|
| Butenes | 7.8 | 1.3 | 0.1 |
| Butanes | 1.9 | 1.7 | 0.1 |
| O₂ | 11.4 | 6.0 | 7.6 |
| H₂O | 5.0 | 12.2 | 0.5 |
| Butadiene | 0.0 | 6.0 | 0.0 |
| CO + CO₂ | 0.0 | 1.2 | 1.5 |
| N₂ | 73.9 | 71.3 | 90.8 |
| Balance | 0.0 | 0.2 | 0.0 |

### Comparative Example 1

The reactor is, as in example 1, firstly flushed with a stream of 1000 standard l/h of nitrogen. After one hour, the measured oxygen content downstream of the reactor and in the recycle gas is less than 0.5% by volume. 620 standard l/h of air and 1000 standard l/h of nitrogen are then introduced into the reactor. The recycle gas stream is set to 2190 standard l/h and kept constant by provision of an appropriately large purge gas stream. After 20 minutes, the oxygen concentration in the recycle gas is 7.9% by volume. The oxygen concentration in the recycle gas stream is thus about as high as in the later steady-state operation, cf. table 1. Supply of air and of nitrogen to the reactor are stopped simultaneously. 225 standard l/h of steam are fed into the reactor. Air and a stream consisting of 80% by volume of butenes and 20% by volume of butanes are then fed into the reactor, with the ratio of air flow to flow of butenes/butanes being regulated in such a way that it is constant at about 3.75. Commencing at a flow of 44 standard l/h of butenes/butanes and 165 standard l/h of air, the flows are increased over a period of one hour at a constant ramp. After one hour, the flow of butenes/butanes is 440 standard l/h and the air flow is 1650 standard l/h. The recycle gas stream is kept constant during the entire start-up operation by separating off an appropriate purge gas stream and is 2190 standard l/h.

The butenes are reacted to an extent of 83% C at a salt bath temperature of 380°. The selectivity of the conversion of the butenes into butadiene is 92%, that to CO and CO₂ together is 5% and that to other secondary components is 3%. The plant is operated for 4 days and a steady state in which the concentrations of the gas components change by not more than 5%/h is established. The concentrations in the steady state upstream and downstream of the reactor and in the recycle gas are shown in table 1. The concentration curve for butanes/butenes (fuel gas), oxygen and the remaining gas components (100%−C_{fuel gas}−C_{O2}) upstream of the reactor (“reactor”) and between the quench and the compression stage (“absorption”) and in the recycle gas (“recycle gas”) is shown together with the explosion diagrams for the reactor (“ex. reactor”) and the absorption column (“ex. absorption”) in FIG. 3. All concentrations are reported in percent by volume. The concentration of the fuel gas is plotted on the ordinate, and the concentration of oxygen is plotted on the abscissa. Immediately before the introduction of fuel gas (butenes and butanes) is commenced, the oxygen concentration upstream and downstream of the reactor, between the quench and absorption column and in the recycle gas is 7.9% by volume. During the time in which the fuel gas flow is increased to the final value, the oxygen concentration in the recycle gas changes only slightly to a final value of about 7.6% by volume. Upstream of the reactor and between the quench and absorption column, the oxygen concentration increases; it can be seen that in this case the distance from the explosive region in the reactor is very small during start-up of the reactor. Safe process operation is difficult to realize in this case.

### Comparative Example 2

The reactor is, as in example 1, firstly flushed with a stream of 1000 standard l/h of nitrogen. After one hour, the measured oxygen content downstream of the reactor and in the recycle gas stream is less than 0.5% by volume. The recycle gas stream is set to 2190 standard l/h and kept constant by branching off an appropriately large purge gas stream. The oxygen content in the recycle gas stream is consequently 0% by volume. The nitrogen stream is stopped and 225 standard l/h of steam are fed into the reactor. Air and a stream consisting of 80% by volume of butenes and 20% by volume of butanes are then fed to the reactor, with the ratio of stream of air to stream of butenes/butanes being regulated so that it is always about 3.75. Commencing with a flow of 44 standard l/h of butenes/butanes and 165 standard l/h of air, the flows are increased over a period of one hour at a constant ramp. After one hour, the flow of butenes/butanes is 440 standard l/h and the air flow is 1650 standard l/h. The recycle gas stream continues to be 2190 standard l/h.

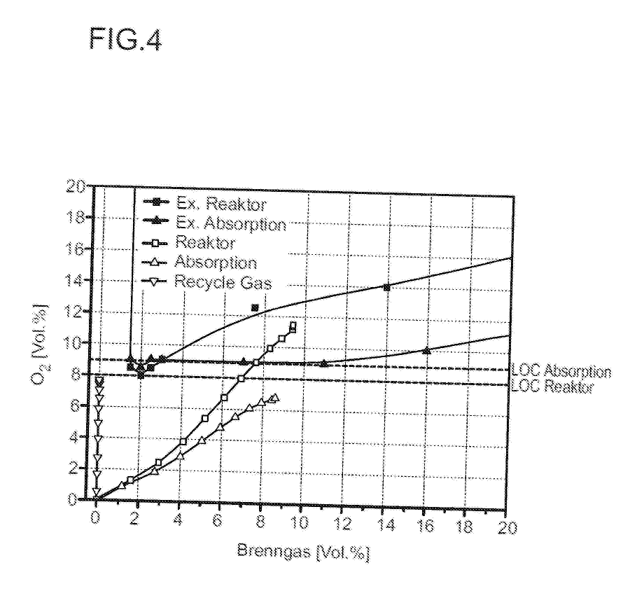

The butenes are reacted to an extent of 83% at a salt bath temperature of 380° C. The selectivity of the conversion of butene into butadiene is 92%, that to CO and CO₂ together is 5% and that to other secondary components is 3%. The plant is operated for 4 days and a steady state in which the concentrations of the gas components change by not more than 5%/h is established. The concentrations in the steady state upstream and downstream of the reactor and in the recycle gas are shown in table 1. The concentration curve for butanes/butenes (fuel gas), oxygen and the remaining gas components (100%−C_{fuel gas}−C_{O2}) upstream of the reactor and between the quench and the compression stage and in the recycle gas is shown together with the explosion diagrams for the reactor (“ex. reactor”) and the absorption column (“ex. absorption”) in FIG. 4. All concentrations are reported in percent by volume. The concentration of the fuel gas is plotted on the ordinate, and the concentration of oxygen is plotted on the abscissa. Immediately before the introduction of fuel gas (butenes and butanes) is commenced, the oxygen concentration upstream and downstream of the reactor (“reactor”), between the quench and the absorption column (“absorption”) and in the recycle gas (“recycle gas”) is less than 0.5% by volume. During the time in which the fuel gas flow is increased to the final value, the oxygen concentration in the recycle gas increases to a final value of about 7.6% by volume. The oxygen concentration also increases upstream of the reactor and between the quench and the absorption column, but without crossing into the explosive region. The disadvantage of this type of start-up is the very low oxygen contents at the beginning of the start-up operation. It is known that the catalyst tends to undergo rapid carbonization in the presence of butenes at low oxygen contents. Carbonization can in the long term lead to loss of the activity of the catalyst and to mechanical destruction thereof.

1.-13. (canceled)

14. A process for preparing butadiene from n-butenes having a start-up phase and an operating phase, wherein the process in the operating phase comprises the steps:
A) provision of a feed gas stream a1 comprising n-butenes;
B) introduction of the feed gas stream a1 comprising n-butenes, of an oxygen-comprising gas stream a2 and of an oxygen-comprising recycle gas stream d2 into at least one oxidative dehydrogenation zone and oxidative dehydrogenation of n-butenes to butadiene, giving a product gas stream b comprising butadiene, unreacted n-butenes, water vapor, oxygen, low-boiling hydrocarbons, high-boiling secondary components, possibly carbon oxides and possibly inert gases;
C) cooling and compression of the product gas stream b and condensation of at least part of the high-boiling secondary components, giving at least one aqueous condensate stream c1 and a gas stream c2 comprising butadiene, n-butenes, water vapor, oxygen, low-boiling hydrocarbons, possibly carbon oxides and possibly inert gases;
D) introduction of the gas stream c2 into an absorption zone and separation of incondensable and low-boiling gas constituents comprising oxygen, low-boiling hydrocarbons, possibly carbon oxides and possibly inert gases as gas stream d from the gas stream c2 by absorption of the C₄-hydrocarbons comprising butadiene and n-butenes in an absorption medium, giving an absorption medium stream loaded with C₄-hydrocarbons and the gas stream d, and recirculation, optionally after separating off a purge gas stream p, of the gas stream d as recycle gas stream d2 to the oxidative dehydrogenation zone;
and the start-up phase comprises the steps:
i) introduction of the oxygen-comprising gas stream and an inert gas stream into the dehydrogenation zone in such a ratio that the oxygen content of the recycle gas stream d2 corresponds to from 30 to 80% of the oxygen content of the recycle gas stream d2 in the operating phase;
ii) setting of the recycle gas stream d2 to at least 70% of the volume flow of the recycle gas d2 in the operating phase;
iii) optional introduction, at an initial oxygen content of the recycle gas stream d2 of from 30 to 80% of the oxygen content of the recycle gas stream d2 in the operating phase, of a steam stream a3 into the dehydrogenation zone;
iv) introduction, at an initial oxygen content of the recycle gas stream d2 of from 30 to 80% of the oxygen content of the recycle gas stream d2 in the operating phase, of an oxygen-comprising gas stream a2′ and a butene-comprising feed gas stream a1′ having a smaller volume flow than in the operating phase in a ratio k=a2′/a1′ and raising of the volume flow of the gas streams a1′ and a2′ until the volume flows of the gas streams a1 and a2 in the operating phase are obtained, with the recycle gas stream d2 being at least 70% and not more than 120% of the volume flow in the operating phase.

15. The process according to claim 14, wherein the ratio k is from 1 to 10.

16. The process according to claim 15, wherein the ratio k is from 1.5 to 6.

17. The process according to claim 14, wherein the recycle gas stream d2 is set in step ii) to from 70 to 120% of the volume flow in the operating phase.

18. The process according to claim 14, wherein the oxygen content of the recycle gas stream d2 in step i) corresponds to from 40 to 70% of the oxygen content of the recycle gas stream d2 in the operating phase.

19. The process according to claim 14, wherein the introduction of the inert gas stream is stopped between step ii) and step iii).

20. The process according to claim 14, wherein the amount of water vapor in the dehydrogenation zone during steps iii) and iv) is from 0 to 20% by volume.

21. The process according to claim 14, wherein the pressure in the dehydrogenation zone during the start-up phase is from 1 to 5 bar.

22. The process according to claim 14, wherein the pressure in the absorption zone during the start-up phase is from 2 to 20 bar.

23. The process according to claim 14, wherein step D) comprises the steps Da) and Db):
Da) separation of incondensable and low-boiling gas constituents comprising oxygen, low-boiling hydrocarbons, possibly carbon oxides and possibly inert gases as gas stream d from the gas stream c2 by absorption of the C4-hydrocarbons comprising butadiene and n-butenes in an absorption medium, giving an absorption medium stream loaded with C4-hydrocarbons and the gas stream d, and
Db) subsequent desorption of the C4-hydrocarbons from the loaded absorption medium stream, giving a C4 product gas stream d1.

24. The process according to claim 23 having the additional steps:
E) separation of the C₄ product stream d1 by extractive distillation using a butadiene-selective solvent into a stream e1 comprising butadiene and the selective solvent and a stream e2 comprising n-butenes;
F) distillation of the stream f2 comprising butadiene and the selective solvent to give a stream g1 consisting essentially of the selective solvent and a butadiene-comprising stream g2.

25. The process according to claim 14, wherein the absorption medium used in step D) is an aromatic hydrocarbon solvent.

26. The process according to claim 14, wherein a shutdown mechanism prevents the oxidative dehydrogenation reactor from being supplied with a feed gas mixture whose composition is explosive, where the shutdown mechanism is configured as follows:
a) an explosion diagram characteristic of the feed gas mixture, in which explosive and nonexplosive compositions are delineated from one another as a function of the composition of the feed gas mixture, is stored in a computer;
b) a data set is determined by measurements of the amount and optionally composition of the gas streams fed into the dehydrogenation zone for producing the feed gas mixture and this data set is transmitted to the computer;
c) a current operating point of the feed gas mixture in the explosion diagram is calculated by the computer from the data set obtained under b);
d) if the distance of the operating point from the closest explosion limit drops below a prescribed minimum value, the supply of gas streams to the dehydrogenation zone is automatically interrupted.
